SEQUENCE LISTING (13 sequences)

SEQ ID NO: 1 - DNA, 1908 bp, Caenorhabditis elegans
atgatcatatgttattcgtgtctaactgtctccattcttctaaccattaaatttgtaccatgtcgatttgctggaattgaacaccaaaatacgaaaagtcgtgtgcatttctcgttgctggatagtagacaagaaaatgacactaatcactttgagatagcagaagcaaagttccagaaaccccacaatgaggaaaacacaataggtacgattacaaaatttgctccatcggtacaagaacaacacagttctgcggtaattccaatgccccactttgaccagaaccggcttgagcaagctcttcggatcaagggctcaattgatggaaccgaagaggctttgtacaggtctctactagatcatactgtttacgaaaaagatgtgaggccatgtatacatcactctcaaccaacaaatgtcacatttggatttcttctcaatcagattgtggaaatggatgaacgaaatcaagctctaacaaccagaagctggctgaatatcaattggatggatcctcgattatcgtggaatgaaagcctttggtctgaaattaaagcaatatatattccacatgcaagaatctggaaacccgatataattctggtaaacaacgctatccgagaatactatgcatccctcgtctccaccgatgtaatggtcacaagtgacggaaacgtgacatggctgttttccgcactatttaggagttcttgtccaatacgggttcgatattatccattcgatgatcaacaatgtgatctgaaatttgcctcctggtcccatgatatcacagaaatcaatctcgggttgaacacggacaaaggggatttgtcaagttatatgaacaacagcgaatttgatcttgtggatatgacggctgttcgagaagttgttacatttccatcggatactaatagtgattggccaataattgtgatacgaatacatatgcatagacgtcctttgttctacgtatttaatcatattgttccttgcgttcttatttcatcaatggcagttcttggtttcctgatgcccccggaaaccggcgagaaaattaacatgatcataacaactttgctctccatgggtgtgtatctgcagtcaatcactgagtcaatacccccaacatccgaaggtgttccattaattggaatgtattacgtatcttctcttcttatggtttgcctagcaacatgcgtaaatgtaatcactcttaacatgcacaggaatggtgcagctaatcagggaaggcacgtgcctgcgtggatgcagaagtggattctggggtacttggccactttcatgagaatgtcaataagagaacccgatagtatagcattgctaaaagcgtcacagagcaaaaagtcaactattcggagaagctcaatacttcgagatttgaaaagggtgaaaaatatgtcaaacgttagagcaaaatcaaaagagcaaaatgcaaatcgagagtgcgagtgcatggacccacttgtgcatatctacgcagagtccatcatgagctgcctggcagcagacacaaaacctatgaacgggtcaactattagagaagattttgcaagtgaaagcacatttcttggacgcgttgttagtgatggcataatgccaagaataagtgcttcatccaactctgtgctgacagaattcgaaacaagatttagacggatattaaaaagggtttaccgaagtcttcagcaacatgaaatacgagaagaaattcttgacgaaagatctcgaattcaatggcagtggcaacaacttgcatctgtcgttgatcgacttttactatgtcttttttgcactgcaacactgttcacaatcatctgcctcctaattgtacctgtagcataccgtgataacgactcaatgttgtcattcctcaattttttctga

SEQ ID NO: 2 - DNA, 1440 bp, Caenorhabditis elegans
atgctcttgcccattttattgcattttttgcttctaatcacccaattaaatggctcaccagcagaagtacggcttatcaatgatcttatgtcaggatatgttcgtgaggaaagaccaacacttgatagttcaaagccagttgttgtcagtttgggagtctttttgcaacagattattaacttgtccgaaaaagaggaacagctggaagtaaatgcctggcttaagttccaatggagagatgaaaatttacgatgggaaccgactgcttatgagaacgtgacagatctaagacatccaccggatgctctatggactcccgatatccttctttataatagtgtcgattcggagtttgattcgtcgtataaagtaaatctggttaattatcatacgggaaacattaattggatgccaccaggaatattcaaagtatcgtgtaaattggatatttattggtttccatttgatgaacaagtttgttattttaagtttggctcatggacgtatactcgtgataagattcaactagaaaagggtgattttgatttctccgagttcattccaaacggggaatggattataatagattatcgaacaaatattactgtgaaacaatatgaatgttgtcccgagcagtatgaagatatcacttttacgctacatttacgacggagaactttatactattccttcaatttaattgctccagttcttttaacaatgatactggttattttgggctttactgttagccctgaaacttgcgaaaaagttggacttcagatctctgtctctcttgccatatgcattttcctcacaataatgagtgaactgacacctcaaacatcagaagctgttccacttcttggagtattcttccacacttgcaacttcatttccgttttagccacttctttcacagtttatgtgcaaagttttcattttcgaaaccaacatgtacacgaacggatggatttctggatgaggttcattctcttggagtggtcaccgtggctattgcgaatgaaaatgccggatagagaaaataactttcagacactgacagaaagttggaagggaaggaatcgaagagaatctatggcaagaacagcgttcgaatatgcagatggaccggttacacagatacattccatgggaattatgttgaaagataattttgaagagcttatttatcaagttaaacaggagaagattgctgatgaaaaaggaattgagagattgcgggtgttacagaagatttacgatcatgtaaagatgatccgagaacatgacgatgacaatgatgaagacagtcgagtagctcttgaatggagatttgctgcaattgtcgtcgatcgtctgtgccttcttgccttctccttactcatcgtcgtcgtctccatcatcattgctttacgtgcaccgtatcttttcgcttaa

SEQ ID NO: 3 - DNA, 1665 bp, Caenorhabditis elegans
atgaggagaaggttcgaaatcggcatcgcattctttttcgcactttttcgagtgatatggacgggtgaccatgaacgtagactatatgcaaaattggcggaaaactacaacaaattggcgagacctgttcgaaatgaaagtgaagctgtagtagttcttcttgggatggattatcaacaaattttggatattgacgaaaaacatcaaataatgaattcaagtgtttggttacggatgtcatggacagatcattacttgacatgggatccatcagagtttggaaatatcaaagaagttcgtttgccaatcaataatatctggaaacctgatgttcttctctacaatagtgttgatcaacagtttgatagtacatggcccgttaatgctgttgttttgtacacgggaaacgtaacgtggattcctccagccatcattcgatcaagttgtgctattgacatagcatattttccatttgatactcaacattgtactatgaagttcggttcctggacatattctggttttttcactgatctcattaacacaacaatatctccagccacttataaaccaaatggagaatgggaattacttggcttaacgtcgcaacgctcgatatttttctatgaatgctgcccggagccatattatgatgtcacgtttactgtttcaattaggaggagaactctctattatggattcaacttattgctcccatgtatgctcatttcctcactggctttgttgagtttcacacttccagctgattgtggagagaaactgaatttaggcgtcacaatcttcatgtctctttgcgtttttatgattatggttgctgaagcaatgcctcaaacaagtgatgcacttccattaattcaaatctatttctcgtgcataatgttccaagttggtgcatcagtggtggccactgtgattgcattgaactttcatcatcgatcaccagaacagtacaagcctatgaacaaatttttgaaaactcttcttctgggctggcttccaacacttcttggcatggaacgtcctgatgttcttgaactttctgtacatggagcacattatgcgtctgacaataaaaaaaaacaacgtcaatacctaatagaagtggagagacatattctaacccgtccaaatggaaatggacattcagcagttgataaagcagtgcatcttgacttatcaactggtaatccacactctgatgctaaaaaatcatcaccttctccaaaacgaacaagtgcttcaataatgggtatgactggattgccaacaactcaaatgaatggagcattggattcttcaattaataaatatacttgtacaaaagttacgcgtccactggaaaacggttcagcaacaataaatcacaaatcatcacctcaaataaatccaatcaataacaataatatctataaatgtgcaaacaaccaaaagactcaattcgaagatcgtcattttcatcatattctgaatgagcttcgtgttatatcagctcgtgtgagaaaagaagaagcaatgcatgcacttcaagctgattggatgtttgcaagtcgagttgtagatcgggtttgttttcttgctttttcagcatttctcttcatgtgcactgctattatttcttataatgccccgcatttatttgtataa

SEQ ID NO: 4 - DNA, 2138 bp, Caenorhabditis elegans
tgtccagtcgacgggccctcaattccccccgtaaatattctttatgatcatatgttattcgtgtctaactgtctccattcttctaaccattaaatttgtaccatgtcgatttgctggaattgaacaccaaaatacgaaaagtcgtgtgcatttctcgttgctggatagtagacaagaaaatgacactaatcactttgagatagcagaagcaaagttccagaaaccccacaatgaggaaaacacaataggtacgattacaaaatttgctccatcggtacaagaacaacacagttctgcggtaattccaatgccccactttgaccagaaccggcttgagcaagctcttcggatcaagggctcaattgatggaaccgaagaggctttgtacaggtctctactagatcatactgtttacgaaaaagatgtgaggccatgtatacatcactctcaaccaacaaatgtcacatttggatttcttctcaatcagattgtggaaatggatgaacgaaatcaagctctaacaaccagaagctggctgaatatcaattggatggatcctcgattatcgtggaatgaaagcctttggtctgaaattaaagcaatatatattccacatgcaagaatctggaaacccgatataattctggtaaacaacgctatccgagaatactatgcatccctcgtctccaccgatgtaatggtcacaagtgacggaaacgtgacatggctgttttccgcactatttaggagttcttgtccaatacgggttcgatattatccattcgatgatcaacaatgtgatctgaaatttgcctcctggtcccatgatatcacagaaatcaatctcgggttgaacacggacaaaggggatttgtcaagttatatgaacaacagcgaatttgatcttgtggatatgacggctgttcgagaagttgttacatttccatcggatactaatagtgattggccaataattgtgatacgaatacatatgcatagacgtcctttgttctacgtatttaatcatattgttccttgcgttcttatttcatcaatggcagttcttggtttcctgatgcccccggaaaccggcgagaaaattaacatgatcataacaactttgctctccatgggtgtgtatctgcagtcaatcactgagtcaatacccccaacatccgaaggtgttccattaattggaatgtattacgtatcttctcttcttatggtttgcctagcaacatgcgtaaatgtaatcactcttaacatgcacaggaatggtgcagctaatcagggaaggcacgtgcctgcgtggatgcagaagtggattctggggtacttggccactttcatgagaatgtcaataagagaacccgatagtatagcattgctaaaagcgtcacagagcaaaaagtcaactattcggagaagctcaatacttcgagatttgaaaagggtgaaaaatatgtcaaacgttagagcaaaatcaaaagagcaaaatgcaaatcgagagtgcgagtgcatggacccacttgtgcatatctacgcagagtccatcatgagctgcctggcagcagacacaaaacctatgaacgggtcaactattagagaagattttgcaagtgaaagcacatttcttggacgcgttgttagtgatggcataatgccaagaataagtgcttcatccaactctgtgctgacagaattcgaaacaagatttagacggatattaaaaagggtttaccgaagtcttcagcaacatgaaatacgagaagaaattcttgacgaaagatctcgaattcaatggcagtggcaacaacttgcatctgtcgttgatcgacttttactatgtcttttttgcactgcaacactgttcacaatcatctgcctcctaattgtacctgtagcataccgtgataacgactcaatgttgtcattcctcaattttttctgattatcaaatacttgtttacatgttcttaatgaaatttgcgaattatggagaatatatttgctagaatcaaattttcgggacttgtgtagtattggctgaaaaatttttatccattttgaacttttgatatgaccctttttggttgcattacgtttatgaccagtttttaaagcctaaaaaaaaaaaa

SEQ ID NO: 5 - DNA, 1503 bp, Caenorhabditis elegans
tgatgctcttgcccattttattgcattttttgcttctaatcacccaattaaatggctcaccagcagaagtacggcttatcaatgatcttatgtcaggatatgttcgtgaggaaagaccaacacttgatagttcaaagccagttgttgtcagtttgggagtctttttgcaacagattattaacttgtccgaaaaagaggaacagctggaagtaaatgcctggcttaagttccaatggagagatgaaaatttacgatgggaaccgactgcttatgagaacgtgacagatctaagacatccaccggatgctctatggactcccgatatccttctttataatagtgtcgattcggagtttgattcgtcgtataaagtaaatctggttaattatcatacgggaaacattaattggatgccaccaggaatattcaaagtatcgtgtaaattggatatttattggtttccatttgatgaacaagtttgttattttaagtttggctcatggacgtatactcgtgataagattcaactagaaaagggtgattttgatttctccgagttcattccaaacggggaatggattataatagattatcgaacaaatattactgtgaaacaatatgaatgttgtcccgagcagtatgaagatatcacttttacgctacatttacgacggagaactttatactattccttcaatttaattgctccagttcttttaacaatgatactggttattttgggctttactgttagccctgaaacttgcgaaaaagttggacttcagatctctgtctctcttgccatatgcattttcctcacaataatgagtgaactgacacctcaaacatcagaagctgttccacttcttggagtattcttccacacttgcaacttcatttccgttttagccacttctttcacagtttatgtgcaaagttttcattttcgaaaccaacatgtacacgaacggatggatttctggatgaggttcattctcttggagtggtcaccgtggctattgcgaatgaaaatgccggatagagaaaataactttcagacactgacagaaagttggaagggaaggaatcgaagagaatctatggcaagaacagcgttcgaatatgcagatggaccggttacacagatacattccatgggaattatgttgaaagataattttgaagagcttatttatcaagttaaacaggagaagattgctgatgaaaaaggaattgagagattgcgggtgttacagaagatttacgatcatgtaaagatgatccgagaacatgacgatgacaatgatgaagacagtcgagtagctcttgaatggagatttgctgcaattgtcgtcgatcgtctgtgccttcttgccttctccttactcatcgtcgtcgtctccatcatcattgctttacgtgcaccgtatcttttcgcttaaaccaaatgccttgagcaatcaaataaaaccatttcatttccaaaaaaaaaaaaaaaaaaaa

SEQ ID NO: 6 - DNA, 1915 bp, Caenorhabditis elegans
tgtttgagcaactctcaatgccacgccaccaaggtcgacaaggatgaggagaaggttcgaaatcggcatcgcattctttttcgcactttttcgagtgatatggacgggtgaccatgaacgtagactatatgcaaaattggcggaaaactacaacaaattggcgagacctgttcgaaatgaaagtgaagctgtagtagttcttcttgggatggattatcaacaaattttggatattgacgaaaaacatcaaataatgaattcaagtgtttggttacggatgtcatggacagatcattacttgacatgggatccatcagagtttggaaatatcaaagaagttcgtttgccaatcaataatatctggaaacctgatgttcttctctacaatagtgttgatcaacagtttgatagtacatggcccgttaatgctgttgttttgtacacgggaaacgtaacgtggattcctccagccatcattcgatcaagttgtgctattgacatagcatattttccatttgatactcaacattgtactatgaagttcggttcctggacatattctggttttttcactgatctcattaacacaacaatatctccagccacttataaaccaaatggagaatgggaattacttggcttaacgtcgcaacgctcgatatttttctatgaatgctgcccggagccatattatgatgtcacgtttactgtttcaattaggaggagaactctctattatggattcaacttattgctcccatgtatgctcatttcctcactggctttgttgagtttcacacttccagctgattgtggagagaaactgaatttaggcgtcacaatcttcatgtctctttgcgtttttatgattatggttgctgaagcaatgcctcaaacaagtgatgcacttccattaattcaaatctatttctcgtgcataatgttccaagttggtgcatcagtggtggccactgtgattgcattgaactttcatcatcgatcaccagaacagtacaagcctatgaacaaatttttgaaaactcttcttctgggctggcttccaacacttcttggcatggaacgtcctgatgttcttgaactttctgtacatggagcacattatgcgtctgacaataaaaaaaaacaacgtcaatacctaatagaagtggagagacatattctaacccgtccaaatggaaatggacattcagcagttgataaagcagtgcatcttgacttatcaactggtaatccacactctgatgctaaaaaatcatcaccttctccaaaacgaacaagtgcttcaataatgggtatgactggattgccaacaactcaaatgaatggagcattggattcttcaattaataaatatacttgtacaaaagttacgcgtccactggaaaacggttcagcaacaataaatcacaaatcatcacctcaaataaatccaatcaataacaataatatctataaatgtgcaaacaaccaaaagactcaattcgaagatcgtcattttcatcatattctgaatgagcttcgtgttatatcagctcgtgtgagaaaagaagaagcaatgcatgcacttcaagctgattggatgtttgcaagtcgagttgtagatcgggtttgttttcttgctttttcagcatttctcttcatgtgcactgctattatttcttataatgccccgcatttatttgtataattttttctaattcaatagagtaagagtcaagaaattcatatctcttgttgcttctttttaaattttacatttagagccaatttgtgattttaagtacaaatgtatatctttatttcgtctttttaaaataacatatacagtttcaattgtttttgctttgttgtacatataaacaattattaaatttaaaaaaaaaaaaaaaaaaaa

SEQ ID NO: 7 - protein, 10 aa, Caenorhabditis elegans
MIICYSCLTV

SEQ ID NO: 8 - protein, 10 aa, Caenorhabditis elegans
MLLPILLHFL

SEQ ID NO: 9 - protein, 10 aa, Caenorhabditis elegans
MRRRFEIGIA

SEQ ID NO: 10 - protein, 635 aa, Caenorhabditis elegans
MIICYSCLTVSILLTIKFVPCRFAGIEHQNTKSRVHFSLLDSRQENDTNHFEIAEAKFQKPHNEENTIGTITKFAPSVQEQHSSAVIPMPHFDQNRLEQALRIKGSIDGTEEALYRSLLDHTVYEKDVRPCIHHSQPTNVTFGFLLNQIVEMDERNQALTTRSWLNINWMDPRLSWNESLWSEIKAIYIPHARIWKPDIILVNNAIREYYASLVSTDVMVTSDGNVTWLFSALFRSSCPIRVRYYPFDDQQCDLKFASWSHDITEINLGLNTDKGDLSSYMNNSEFDLVDMTAVREVVTFPSDTNSDWPIIVIRIHMHRRPLFYVFNHIVPCVLISSMAVLGFLMPPETGEKINMIITTLLSMGVYLQSITESIPPTSEGVPLIGMYYVSSLLMVCLATCVNVITLNMHRNGAANQGRHVPAWMQKWILGYLATFMRMSIREPDSIALLKASQSKKSTIRRSSILRDLKRVKNMSNVRAKSKEQNANRECECMDPLVHIYAESIMSCLAADTKPMNGSTIREDFASESTFLGRVVSDGIMPRISASSNSVLTEFETRFRRILKRVYRSLQQHEIREEILDERSRIQWQWQQLASVVDRLLLCLFCTATLFTIICLLIVPVAYRDNDSMLSFLNFF

SEQ ID NO: 11 - protein, 479 aa, Caenorhabditis elegans
MLLPILLHFLLLITQLNGSPAEVRLINDLMSGYVREERPTLDSSKPVVVSLGVFLQQIINLSEKEEQLEVNAWLKFQWRDENLRWEPTAYENVTDLRHPPDALWTPDILLYNSVDSEFDSSYKVNLVNYHTGNINWMPPGIFKVSCKLDIYWFPFDEQVCYFKFGSWTYTRDKIQLEKGDFDFSEFIPNGEWIIIDYRTNITVKQYECCPEQYEDITFTLHLRRRTLYYSFNLIAPVLLTMILVILGFTVSPETCEKVGLQISVSLAICIFLTIMSELTPQTSEAVPLLGVFFHTCNFISVLATSFTVYVQSFHFRNQHVHERMDFWMRFILLEWSPWLLRMKMPDRENNFQTLTESWKGRNRRESMARTAFEYADGPVTQIHSMGIMLKDNFEELIYQVKQEKIADEKGIERLRVLQKIYDHVKMIREHDDDNDEDSRVALEWRFAAIVVDRLCLLAFSLLIVVVSIIIALRAPYLFA

SEQ ID NO: 12 - protein, 554 aa, Caenorhabditis elegans
MRRRFEIGIAFFFALFRVIWTGDHERRLYAKLAENYNKLARPVRNESEAVVVLLGMDYQQILDIDEKHQIMNSSVWLRMSWTDHYLTWDPSEFGNIKEVRLPINNIWKPDVLLYNSVDQQFDSTWPVNAVVLYTGNVTWIPPAIIRSSCAIDIAYFPFDTQHCTMKFGSWTYSGFFTDLINTTISPATYKPNGEWELLGLTSQRSIFFYECCPEPYYDVTFTVSIRRRTLYYGFNLLLPCMLISSLALLSFTLPADCGEKLNLGVTIFMSLCVFMIMVAEAMPQTSDALPLIQIYFSCIMFQVGASVVATVIALNFHHRSPEQYKPMNKFLKTLLLGWLPTLLGMERPDVLELSVHGAHYASDNKKKQRQYLIEVERHILTRPNGNGHSAVDKAVHLDLSTGNPHSDAKKSSPSPKRTSASIMGMTGLPTTQMNGALDSSINKYTCTKVTRPLENGSATINHKSSPQINPINNNNIYKCANNQKTQFEDRHFHHILNELRVISARVRKEEAMHALQADWMFASRVVDRVCFLAFSAFLFMCTAIISYNAPHLFV

SEQ ID NO: 13 - protein, 4 aa, Artificial Sequence
  Derived from C. elegans genome
DIAY

(19) United States
(12) Patent Application Publication
(10) Pub. No.: US 2003/0186370 A1
(43) Pub. Date: Oct. 2, 2003

(54) 5ht3 receptors of nematodes, polynucleotide molecules encoding same, and antagonists thereof

(76) Inventors: Stephen Charles Trowell, Oxley (AU); Mira Dumancic, Oxley (AU); Chunyan Llao, Red Hill (AU); Peter Easr, O'Connor (AU)

Correspondence Address: MCDERMOTT WILL & EMERY, 600 13TH STREET, N.W., WASHINGTON, DC, 20005-3096, US

(21) Appl. No.: 10/203,968

(22) PCT Filed: Feb. 15, 2001
(86) PCT No.: PCT/AU01/00150

(30) Foreign Application Priority Data
Feb. 15, 2000 (AU) PQ 5634

(51) Int. Cl. C12P 21/02; C12N 5/06; C07K 14/705; A01N 61/00; G01N 33/53

(52) U.S. Cl. 435/69.1; 435/320.1; 435/325; 435/7.1; 514/12; 530/350; 536/23.5; 514/1

(57) ABSTRACT

Invertebrate 5-HT₃ receptors, especially from the nematode Ceanorhabditis elegans, and polynucleotide molecules encoding same are disclosed. The receptors and polynucleotide molecules may be used in assays to identify and/or assess candidate compounds for use as nematicidal, insecticidal and/or other pesticidal use.

### FIELD OF THE INVENTION

The present invention relates to the identification of compounds for the control of nematodes, insects and other invertebrate pests. More particularly, the present invention relates to the isolation and cloning of polynucleotide molecules encoding nematode 5-HT₃ receptors. The receptors may be used in assays for the identification and/or assessment of candidate nematicidal, insecticidal and other pesticidal compounds.

### BACKGROUND TO THE INVENTION

Many species of nematodes are parasites of considerable medical. veterinary and agricultural significance. For example. nematodes of the Orders Strongylida, Strongyloides, Ascaradida, Oxyurida and Trichocephalida include many species that cause disease in humans, sheep, cattle, pigs and other species. Further, nematodes of the Orders Tylenchida and Aphelenchida, and others, include species which are parasitic of important crop plants and fungi.

It has been conservatively estimated that plant parasitic nematodes cause US $77 billion worth of damage to major food crops annually [Evans, K. and Haydock, P. 1999. Control of plant parasitic nematodes. Pesticide Outlook. 107-111. There are, unfortunately, very few control options for plant parasitic nematodes. Fumigants such as methyl bromide are generally being withdrawn from sale and use, because of their detrimental effects on the ozone layer, whilst the remaining available agents are among the most toxic and undesirable pesticides in current use.

Animal parasitic nematodes infect humans, companion animals and livestock and cause serious morbidity and economic loss worldwide. This group of parasites includes hookworms and roundworms. They can cause anaemia, loss of weight, hyperimmune reactions and other complications, including death of livestock. There are currently a small number of human and veterinary drugs available for their treatment but, particularly in the veterinary field, the efficacy of a number of existing drugs is dropping because of resistance development.

For the reasons described, there is an ongoing need for the identification of new nematicidal compounds.

Pharyngeal pumping is the basis of nematode feeding and the ability of nematodes to maintain their “hydrostatic skeleton” [Brownlee, D. J. A. et al. 1997. Actions of the anthelmintic ivermectin on the pharngeal muscle of the parasitic nematode. Ascaus suum. Parasitology, 115: 553-561]. The pharyngeal pump of nematodes is already a well-established target organ for anthelminthics and nematicidal agents. In particular, inhibition of pharyngeal pumping is a major mode of action of ivermectin, an extremely successful modern nematicide and insecticide. Ivermectin acts on inhibitory glutamate receptors present in the pharynx and other tissues of nematodes and insect [Brownlee. D. J. A. et al. 1997, supra]. Identification of new molecular targets in the nematode pharynx would greatly facilitate the discovery process for nematicidal compounds and insecticidal compounds since knowledge of a molecular target can assist in lead compound choice and/or design. Further, for targets representing molecular receptors, the possibility of isolating the receptor gene offers the prospect of using cloned receptors to screen natural product collections and synthetic compound libraries. Active molecules recovered from these screens may have utility in controlling nematodes, insects and other pests. Examples of this include the macrocyclic lactone nematicides (avermectins) which were originally registered as anthelminthics but are now being used increasingly as insecticides.

The damage caused by insect pests is better known and characterised than that caused by nematodes. For example. the worldwide market for chemical insecticides is about US $12 billion. mostly in crop protection, but also in animal and public health. The market is growing at about 5% p.a. These costs of control are only a fraction of the economic costs of losses to crops and livestock worldwide. The role of insects in vectoring major diseases to humans is also well-known. In particular, sucking plant pests such as aphids and plant-hoppers are second only to caterpillars in their economic importance and their value as a market for insecticides. They are particularly important in Europe and Asia. Whilst there are some existing pesticides active against these pests, a number of them are highly toxic and development of resistance is also causing problems. Thus, there is a great need for new classes of insecticides active on these pests.

Also, insects with piercing and sucking mouthparts are the main vectors of diseases to humans and livestock. These vectors include mosquitoes (e.g. malaria, Japanese encephalitis, dengue fever etc.), higher flies (e.g. onchocerciasis) and true bugs (e.g. trypanosomiosis). Existing control measures are increasingly reliant on pesticides (e.g. permethrin-treated mosquito nets), because of the absence or failure of drug treatments. Therefore, there is also a need for new classes of insecticides active against these pests.

Serotonin (5-Hydroxytryptamine, 5-HT) is known to have a number of profound effects on the behaviour of Caenorhabditis elegans and other nematodes. In C. elegans, exogenously applied 5-HT results in reduced locomotion, increased pharyngeal pumping, increased egg-laving and decreased defaecation. It is also involved in male mating behaviour. These effects of exogenous 5-HT are believed to occur because 5-HT is a natural nematode neurotransmitter that serves these behaviours. For example, two serotonergic neurones (NSM) are located over the pharynx whilst the HSNL and HSNR serotonergic neurones connect with the vulva. However, it is quite likely, based on the known biology of 5-HT in vertebrates that each of these behaviours is controlled by action on different receptors present in different cells.

Vertebrate serotonin receptors are known to fall into two distinct multigene superfamilies. One of these, the rhodopsin/β-adrenergic receptor superfamily, includes 7-transmemberane G-protein-linked receptors of the 5-HT₁, 5-HT₂, 5-HT₄, 5-HT₅, 5-HT₆, and 5-HT₇ classes. Receptors of the 5-HT₃ class belong to the nicotinic-acetylcholine receptor (nAChR), GABA-, glycine- and glutamate-gated ion channel superfamily and are pentameric, 4-membrane-spanning ligand-gated ion channels. It is generally observed that physiologically expressed pentameric receptors of this family comprise two or more types of subunits, with each type being the product of a distinct gene. While functioning ion channels may be obtained experimentally with a pentamer composed of identical subunits, these do not behave identically with respect to their channel conductance properties, to the heteropentameric ion channel that is present in vivo. In the case of the mammalian 5-HT₃ gated ion-channel, faithful electrophysiology has only been obtained with a heteromeric ion channel containing both 5-HT_{3A} and 5-HT_{3B} subunits [Davies, P. A. et a]. 1999. The 5-HT_{3B} subunit is a major determinant of serotonin receptor function. Nature. 397, 359-3631. The mammalian 5-HT₃ receptors are known to form channels that gate the passage of cations across the cell membrane and when activated they tend to excite the cell. In this respect, as in many others, their closest relatives are the nicotinic acetylcholine receptors. GABA_{A}-gated, glycine-gated, and the invertebrate-specific glutamate-gated ion channels all gate the passage of anions and their activation generally hyperpolarises the cell membrane. It should be noted that, although ligand-gated ion-channels are often heteropentamers (i.e. composed of five receptor subunits with the subunits being the products of two or more genes. as was unequivocally demonstrated for the mammalian 5-HT₃ receptor [Davies, P. A. et al., 1999, supra], it is possible to form functional channels with only a single subunit and, furthermore, expressed single subunits bind serotonergic ligands with expected authentic pharmacology [(e.g. Maricq, A. V. et al., 1991. Primary structure and functional expression of the 5-HT₃ receptor, a serotonin-gated ion channel, Science, 254)]. This means that it is possible with known 5-HT₃ receptors to perform functional and radioligand binding screens for agonists and antagonists using expressed single subunits.

In the case of the 7-transmembrane 5-HT receptors, a number of invertebrate homologues have now been cloned. These have significant sequence differences from the vertebrate examples but are nevertheless interpretable within the molecular phylogeny generated for the vertebrate receptors. To the extent that they have been studied there are also some pharmacological differences between vertebrate and invertebrate receptors.

Until now, there has been no report of the cloning of a cationic 5-HT₃ receptor subunit from an invertebrate species. One electrophysiological study of the snail Helix asperse, has been reported which measured a channel with conductance properties and serotonergic pharmacology indicative of a 5-HT₃ receptor [Green. K. A. et al. 1996. Ligand gated ion channels opened by 5-HT in molluscan neurones. Brit. J. Pharmacol. 119: 602-6081]. However, one of the most eminent researchers in the field of C. elegans neurobiology has stated that “[t]here are no sequences in the C. elegans database with significant similarity to the ionotropic serotonin receptor subclass 5-HT₃” [Niacaris, T., and Avery L., Expression patterns of candidate serotonin receptors. The Worm Breeder's Gazette. 15:20, 19981]. The present inventors have now isolated from the invertebrate species, C. elegans, three polynucleotide molecules encoding 5-HT receptor subunits which, based on sequence motif analysis and overall homology, show them to be 5-H₃ receptor subunits. The present inventors have also obtained pharmacological evidence indicating that nematode pharyngeal pumping (which is known to be involved in nematode feeding and maintenance of internal hydrostatic pressure) is under the control of a receptor with 5-HT₃ characteristics, and furthermore, that double stranded RNA-mediated gene suppression of one of these genes reduces the pharyngeal response to exogenous serotonin. The cloning of the disclosed C. elegans 5-HT₃ receptor subunits is therefore of considerable importance for the identification of novel nematicidal compounds.

Very recently, Ranganathan, R. et al. (2000, MOD-1 is a serotonin-gated chloride channel that modulates locomotary behaviour in C. elegans. Nature. 408: 470-475) have described a novel serotonin-gated ion channel from the nematode C. elegans. This seems to control nematode locomotion in response to food, gates anions, unlike all other known 5-HT receptors, and has novel pharmacology in that it does not respond to 5-HT₃-specific inhibitors but does respond to some other serotonergic agents. These results indicate the existence of a second class of serotonin-gated ion-channels distinct from the class that includes ⁵-HT₃A and 5-HT_{3B}. The results of Ranganathan et al. do not indicate whether there is a 5-HT₃ receptor in C. elegans or other invertebrates.

### DISCLOSURE OF THE INVENTION

Thus, in a first aspect, the present invention provides an isolated polynucleotide molecule encoding an invertebrate 5-HT₃ receptor subunit consisting of a nucleotide sequence which substantially corresponds to any one of those shown as SEQ ID NO: 1-6, or which shows greater than 75% (more preferably, greater than 85%, most preferably greater than 95%) homology to any or all of the nucleotide sequences shown as SEQ ID NO: 1-6.

The polynucleotide molecule of the first aspect, may be operably linked to nucleotide sequence elements necessary for, or to enhance, expression. For instance, the polynucleotide molecule may be operably linked to any suitable promoter sequence (e.g. constitutive or inducible promoters), enhancer sequence or other element which regulates expression. Conveniently, the polynucleotide molecule of the first aspect may be introduced into an expression cassette or vector for expression of the encoded 5-HT₃ receptor subunit.

Thus, in a second aspect, the present invention provides an expression cassette or an expression vector comprising a polynucleotide molecule of the first aspect

In a third aspect, the present invention provides a mammalian, insect, plant, yeast or bacterial host cell transformed with the expression cassette or vector of the second aspect.

The host cell of the third aspect may be used to express one or more types of 5-HT₃ receptor subunits (e.g. ⁵-HT_{3A} and 5-HT_{3B} receptor subunits) to allow assembly within said cell of homomeric or heteromeric 5-HT₃ receptors. For the production of heteromeric 5-HT₃ receptors, the host cell is transformed with two or more expression cassettes or vectors, wherein each comprises a polynucleotide molecule encoding a different 5-HT₃ receptor subunit. Alternatively, a single expression vector might be used which comprises two or more polynucleotide molecules each encoding a different 5-HT₃ receptor subunit.

Preferably, said host cell expresses the polynucleotide molecule(s) such that the 5-HT₃ receptors are expressed onto the surface of a host cell.

In a fourth aspect, the present invention provides a method of producing 5-HT₃ receptors, comprising culturing the host cell of the third aspect under conditions enabling the expression of the polynucleotide molecule(s) and, optionally, recovering the expressed receptors.

Preferably, the host cell is mammalian or of insect origin. Where the cell is mammalian, it is presently preferred that it be a COS cell. Chinese hamster ovary (CHO) cell or human embryonic kidney 293 cell. Where the cell is of insect origin, it is presently preferred that it be an insect Sf9 cell.

In a preferred embodiment, the 5-HT₃ receptors are expressed onto the surface of the host cell.

In a fifth aspect, the present invention provides an invertebrate 5-HT₃ receptor comprising at least one subunit which is characterised by an N-terminal amino acid sequence selected from the following:

MIICYSCLTV (SEQ ID NO: 7), MLLPILLHFL (SEQ ID NO: 8) or MRRRFEIGIA (SEQ ID NO: 9),

or a functionally equivalent fragment of said receptor, in a substantially pure form.

Preferably, the at least one subunit has an amino acid sequence substantially corresponding to that shown as SEQ ID NO: 10, 11 or 12.

In a sixth aspect, the present invention provides an assay for identifying and/or assessing nematicidal compounds, said assay comprising contacting a 5-HT₃ receptor or a functionally equivalent fragment thereof according to the fifth aspect, or a cell transfected with and expressing 5-HT₃ receptors from one or more expression cassette(s) or vector(s) of the third aspect, with a candidate nematicidal compound under conditions enabling the activation of 5-HT receptors. and detecting an increase or decrease in activity of said 5-HT₃ receptor(s) or functionally equivalent fragment thereof.

An increase or decrease in activity of the 5-HT₃ receptor(s) or functionally equivalent fragment thereof may be detected by measuring changes in cell membrane potential or Ca²⁺ levels.

The step of contacting may involve contacting the 5-HT₃ receptor(s) or functionally equivalent fragment thereof simultaneously with the candidate nematicidal compound and a serotonergic ligand.

In a seventh aspect, the present invention provides an assay for identifying and/or assessing nematicidal compounds, said assay comprising contacting a 5-HT₃ receptor or a functionally equivalent fragment thereof according to the fifth aspect, or a cell transfected with and expressing 5-HT₃ receptors from one or more expression cassette(s) or vector(s) of the third aspect, with a predetermined amount of a suitably labelled serotonergic ligand together with a predetermined amount of a candidate nematicidal compound under conditions wherein said serotonergic ligand and said candidate nematicidal compound competitively bind to the 5-HT₃ receptor(s) or functionally equivalent fragment thereof. and determining the amount of bound and/or unbound labelled serotonergic ligand.

The determined amount of bound and/or unbound labelled serotonergic ligand enables a calculation of the relative binding affinity of the candidate nematicidal compound to the 5-HT₃ receptor(s) or functionally equivalent fragment thereof.

The suitably labelled serotonergic ligand is preferably 5-hydroxytryptamine (5-HT). The serotonergic ligand may be labelled with, for example. any of radioisotopes (e.g. H³), enzymes, biotin/avidin, a fluorescent molecule or a chemiluminescent molecule.

In the assay of the seventh aspect, the 5-HT₃ receptor, functionally equivalent fragment, or cell expressing 5-HT₃ receptors are preferably anchored to a support (e.g. the well surfaces of a 96-well plate). This allows unbound serotonergic ligand to be readily washed away by methods routine in the art.

In addition, the polynucleotide molecule of the first aspect, especially those comprising all or, at least, a 10 nucleotide part of any of the nucleotide sequences shown as SEQ ID NO: 1-6, may be used to probe for polynucleotide sequences encoding homologous 5-HT₃ receptor subunits from other species.

Accordingly, in a further aspect, the present invention provides a method for identifying a polynucleotide sequence encoding a 5-HT₃ receptor subunit, the method comprising exposing a candidate polynucleotide to a sequence which comprises at least 10 nucleotides of a nucleotide sequence as shown in SEQ ID NO: 1-6. Preferably, the 5-HT₃ receptor subunit can be isolated from an invertebrate.

Such probes may be prepared using routine molecular cloning techniques [as described in, for example, Sambrook, J. et al. 1989. Molecular cloning: a laboratory manual, 2nd edition, Vol. 1-3, Cold Spring Harbor Laboratory Press, Cold Spring Harbor] of a polynucleotide molecule according to the first aspect or a part thereof into a multiple cloning site of a suitable bacterial plasmid (e.g. a multicopy bacterial plasmid such as those of the pUC series including pBlueScript (Stratagene, La Jolla, Calif.), followed by transformation of a bacterial cell line (e.g. DH10B; Life Technologies, Grand island, N.Y.) by well known electroporation techniques. Growth of the host bacterial cell line to high density in small scale liquid culture and isolation of plasmid DNA by alkaline lysis followed by phenol chloroform purification may be conducted as described in Sambrook, J. et al. 1989, supra] (so-called mini-plasmid preparation) or by any one of a number of commercially available kits based on solid-phase adsorption of plasmid DNA followed by selective elution (e.g. “ultraclean MiniPlasmid Prep Kit” Mo Biol Laboratories Inc., Solana Beach, Calif.). Subsequently, the cloned polynucleotide molecule or a part thereof for use as probes, may be excised from the plasmids by restriction endonuclease digestion as is well known in the art. Alternatively, the probes may be generated by polymerase chain reaction (PCR) amplification using a polynucleotide molecule according to he first aspect or a part thereof as template DNA and two PCR primers of suitable length (e.g. 15-30 nucleotides), one of which is homologous to the 5′ terminus of the polynucleotide molecule or part thereof and the other being homologous to the 3′ terminus of the polynucleotide molecule or part thereof. PCR may conveniently be performed according to any of the methods well known in the art (e.g. as described in Innis, M. A. et al. 1990. PCR Protocols: A Guide to Methods and Applications. Academic Press, San Diego, Calif.).

Purification of the probes may be achieved by size fractionation on a low-melting point agarose gel, followed by detection of the band by ethidium bromide staining or staining with any other fluorescent nucleic acid dye under ultraviolet illumination, excision of the agarose region containing the probes, and recovery of the probes from the agarose matrix by melting and selective precipitation of DNA [e.g. Sambrook, J. et al. 1989, supra] or by chemical solubilisation of the agarose followed by adsorption of the probes using glass milk or any one of a number of commercially available kits based on solid-phase recovery of DNA from agarose gel matrices (e.g. QIAquick PCR purification kit QIAGEN GmbH, Hilden, Germany).

Labelling of the probes can be achieved by any of the methods well known in the art. including nick translation using a P³² label, random-prime labelling with P³² using, for example, the Giga Prime DNA-Labelling Kit (GeneWorks, Adelaide) or labelling with biotin or digoxigenin, or by tagging the probes with a hapten which is detectable using an enzymatic label such as peroxidase, HRP or luciferase to generate a coloured product or chemiluminescence or other luminescent signal.

Target DNA (e.g. cDNA and genomic libraries) for probing with the probes may be prepared from those species from which it is desired to obtain polynucleotide sequences homologous to a polynucleotide molecule of the present invention starting with approximately 100 mg (or more) of tissue which is derived from an animal or plant parasitic nematode species (e.g. Haemonchus contortus, Ascans suum, Ascazidia galli, Pratylenchus sp, Globodera sp., Meloidogyne incognita, or from any species of insect including those that have pest status in the orders Lepidoptera (e.g. Helicoverpa sp, Heliothis sp), Diptera (e.g. Lucilia sp, Simulium sp, Anopheles sp. Culex sp. Aedes sp.), Hemiptera (e.g. Myzus sp, Aphis sp), or tissue from any other invertebrate, particularly those with piercing and/or sucking mouthparts and a mode of feeding or attachment to the host that involves sucking or pumping of the alimentary canal). The starting tissue may comprise the whole organism or it may comprise the pharynx with its associated neural structures, or the entire alimentary canal with its associated neural structures. Ideally, the starting tissue will be obtained from a life-stage known to express a 5-HT₃ receptor. This information may be obtained by RNA blotting analysis of a range of life-stages using the probes, but if this information is unavailable or unobtainable then it is convenient and possible to use tissue from mixed life-stages as the starting material.

mRNA for the production of a cDNA library is conveniently prepared from the source tissue using a range of commercially available kits such as the QuickPrep Micro RNA Purification Kit (Amersham Pharmacia Biotech Inc., Piscataway, N.J.). Alternatively, traditional methods such as that of Chomczvnski P. and Sacchi N. (1987, Single-step method of RNA isolation by acid guanidium thiocyanate-phenol-chloroform extraction. Anal Biochem. 162: 156-159) may be used for preparation of total RNA followed by purification of mRNA by oligo-dT cellulose chromatography (Sambrook J. et al., 1989, supra). cDNA may also conveniently be prepared from mRNA using many commercially available kits such as the TimeSaver cDNA Synthesis Kit (Amersham Pharmacia Biotech Inc.. Piscataway. N.J.) and the cDNA pool that can be generated is inserted into commercially available lambda arms by conventional ligation techniques. A number of precut variants of lambda phage are available commercially. One suitable variant is lambda gt10-NotI, phosphatased from Promega Corporation (Madison, Wis.). Others include lambda-Zap (Stratagene, La Jolla, Calif.) or lambda-Excel (Amersham Pharmacia Biotech Inc., Piscataway, N.J.). The pool of lambda phage DNA with the the cDNA inserts can then be assembled into a library of infectious phage particles by mixing with commercially available lambda packaging extracts such as MaxPlax packaging extracts (Epicentre Technologies, Corporation. Madison, Wis.). The cDNA library is then available for screening. It is also possible to produce cDNA expression libraries by minor variations of these procedures (Sambrook. J. et al. 1989, supra).

Recovery of homologous polynucleotide sequences may be achieved as follows.

Library Screening

Once libraries have been constructed they may be used to infect Escherichia coli cells of a line supporting lytic infection (e.g. in the case of lambda strain lambda gt10 the strain used would be C600hfl Promega, Corporation, Madison, Wis.) which are plated out on a suitable agar (Sambrook, J. et al. 1989. supra). Plaque lifts may be taken onto nitrocellulose or nylon (e.g. Hybond N+, Amersham Pharmacia Biotech Inc., Piscataway, N.J.) or other suitable filters. Assemblages of the order of 50,000-100,000 or more independent phage from such a cDNA may be readily screened at a single time. To do this, the filters are probed with the labelled probes. Then to recover polynucleotide sequences encoding homologous receptor subunits from species other than C. elegans it is necessary to perform the probing and washing procedures at range of stringencies including low stringency. Therefore, probing and washing is conducted in the range of 0.1-5 times SSC (Sambrook, J. et al. 1989. supra) and in the temperature range 65-45° C. To ensure that homologous polynucleotide sequences are recovered from distantly related nematode species a method of phylogenetic walking may be employed. This involves using the relevant sequence from C. elegans as a probe to recover the homologous sequence from a phylogenetically, relatively-closely related species, for example by preparing and screening a library from another rhabditid nematode such as Steinernema carpocapsae, then by repeating the steps described above to prepare a library from a more distantly related nematode for example a strongylid nematode such as Haemonchus contortus and use the C. elegans probes and probes made from the homologous sequence recovered from S. carpocapsae to screen the H. contortis library and recover the homologous receptor polynucleotide sequence from the latter species. This process may be repeated to isolate the homologous polynucleotide sequence from any nematode species by reiterating the steps of probe generation. cDNA library construction and screening of the library as described above.

Polymerase Chain Reaction Methods

An alternative approach to isolating, from a second species, polynucleotide sequences encoding homologous receptor subunits is to use the polymerase chain reaction (PCR) to amplify homologous probes from the second species and then to label and use these probes to isolate clones from the appropriate cDNA library, as described above. In such a process, degenerate PCR primers are designed to include all possible nucleotide combinations encoding the known amino acids at highly or moderately well-conserved positions within the sequence. Identification of such highly conserved or moderately conserved positions may be achieved by comparing each of the amino acid sequences shown as SEQ ID NO: 10, 11 and 12 with the amino acid sequences of homologous 5-HT₃ receptors, such as those shown in FIG. 3. In addition, any other homologous invertebrate sequences may be included in such a comparison, such as published expressed sequence tags for homologous cDNAs identifiable by BLAST-searching-of the publicly available nucleic acid databases such as the GenBank and EMBL databases and homologous sequences recovered by BLAST searching of the other invertebrate genome databases, in particular the Drosophila melanogaster genome database. Such comparisons are made using multiple alignment algorithms, for example the “pileup” “clustalw” and “eclustalw” algorithms available within the GCG sequence analysis package, written by the Genetics Computing Group at the University of Wisconsin. Similar algorithms are available for most major computing platforms.

Homologous polynucleotide molecules may be used to express 5-HT₃ receptor subunits from other invertebrate species, which can be employed in assays akin to those of the seventh and eighth aspects.

Thus, in an eighth aspect, the present invention provides an assay for identifying and/or assessing nematicidal, insecticidal and/or other pesticidal compounds, said assay comprising; (i) isolating a polynucleotide molecule from an invertebrate other than C. elegans wherein said polynucleotide molecule comprises a nucleotide sequence which encodes a 5-HT₃ receptor subunit and represents a homologue of any or all of the nucleotide sequences shown as SEQ ID NO: 1-6, (ii) expressing said polynucleotide molecule to produce 5-HT₃ receptors or functionally equivalent fragments thereof, (iii) contacting at least one of said produced 5-HT₃ receptors or functionally equivalent fragments thereof to a candidate nematicidal, insecticidal and/or other pesticidal compound under conditions enabling the activation of 5-HT receptors and (iv) detecting an increase or decrease in activity of said produced 5-HT₃ receptor(s) or functionally equivalent fragment(s) thereof.

The 5-HT₃ receptorts) or functionally equivalent fragment(s) thereof mentioned in step (iii) may be present on the surface of a cell (e.g. a host cell expressing the polynucleotide molecule mentioned in step (i)), or a cell lysate (e.g. a lysate of a host cell expressing the polynucleotide molecule mentioned is step (i)), or may be in a substantially purified form.

In a ninth aspect, the present invention provides an assay for identifying and/or assessing nematicidal, insecticidal and/or other pesticidal compounds, said assay comprising; (i) isolating a polynucleotide molecule from an invertebrate other than C. elegans wherein said polynucleotide molecule comprises a nucleotide sequence which encodes a 5-HT₃ receptor subunit and represents a homologue of any or all of the nucleotide sequences shown as SEQ ID NO: 1-6, (ii) expressing said polynucleotide molecule to produce 5-HT₃ receptors or functionally equivalent fragments thereof, (iii) contacting at least one of said produced 5-HT₃ receptors or functionally equivalent fragments thereof with a predetermined amount of a suitably labelled serotonergic ligand together with a predetermined amount of a candidate nematicidal, insecticidal and/or other pesticidal compound under conditions wherein said serotonergic ligand and said candidate compound competitively bind to the 5-HT₃ receptor(s) or functionally equivalent fragment(s) thereof, and (iv) determining the amount of bound and/or labelled serotonergic ligand.

In a tenth aspect, the present invention provides a nematicidal compound identified by the assay of the seventh or eighth aspects.

In an eleventh aspect, the present invention provides a nematicidal, insecticidal and/or other pesticidal compound identified by the assay of the ninth or tenth aspects.

In a further aspect, the present invention provides a method of killing a helminth (e.g. a nematode, cestode or other flatworms), said method comprising exposing said helminth to an effective amount of a compound which alters the activity of a 5-HT₃ receptor of said helminth.

Preferably, said compound inhibits stimulation of the 5-HT₃ receptor by a serotonergic ligand. An effective amount of said compound may be in the range of 100 μM or less, preferably 10 μM or less, more preferably 1 μM or less at the whole organism level. The compound may be provided in combination with a veterinary- or pharmaceutically-acceptable carrier, or any other carrier as may be appropriate.

In a yet further aspect, the present invention provides a composition for killing a helminth comprising an effective amount of a compound which alters the activity of a 5-HT₃ receptor of a helminth, wherein said compound is not ondansetron or tropanyl dichlorobenzoate.

It has also been found that effective insecticides against certain insects (e.g. sucking insects such as aphids or other insects with muscular feeding mechanisms) are provided by compounds which alter the activity of a 5-HT₃ receptor of such insects.

Thus, in still further aspect, the present invention provides a method of killing an insect, said method comprising exposing said insect to an effective amount of a compound which alters the activity of a 5-HT₃ receptor of said insect.

Preferably, said compound inhibits stimulation of the 5-HT₃ receptors by a serotonergic ligand. An effective amount of said compound may be in the range of 100 μM or less, preferably 10 μM or less, more preferably 1 μM or less at the whole organism level. The compound may be provided in combination with an agriculturally-acceptable carrier.

In yet a still further aspect, the present invention also provides an insecticidal composition comprising an effective amount of a compound which alters the activity of a 5-HT₃ receptor of an insect, wherein said compound is not ondansetron or tropanyl dichlorobenzoate.

Reference to percent homology made in this specification have been calculated using the BLAST program blastn as described by Altschul, S. F. et al. 1997 Capped BLAST and PSI-BLAST: a new generation of protein database search programs, Nucleic Acids Research, Vol. 25, No. 17, pp. 3389-3402 (1997).

The term “5-HT₃ receptor” as used herein refers to a receptor having one or more of the following features (1)-(3):

(1) Is a serotonin-gated molecular ion-channel which gates the conductance of cations and is composed of five receptor subunits each of which has a nicotinicoid transmembrane topology (N-terminus, large extracellular domain, 3 transmembrane helices, large intracellular domain, 1 transmembrane helix, C-terminus).

(2) If it is a mammalian 5-HT₃ receptor, is composed of subunits with a high degree of homology to other known mammalian ⁵-HT_{3A} or 5-HT_{3B} subunits. If it is an invertebrate 5-HT₃ receptor, is composed of subunits with a higher level of amino acid homology to mammalian 5-HT₃ receptor subunits than to known mammalian nicotinic acetylcholine receptor subunits.

(3) Has a characteristic pharmacological profile in that it binds to a subset of known 5-HT₃-specific agonists and antagonists with greater selectivity than it binds to agonists and antagonists of other 5-HT receptor classes.

The term “serotonergic ligand” as used herein refers to any compound that selectively binds to one or more subclasses of serotonin receptor. It may activate the receptor (agonists) or prevent other ligands binding without itself activating the receptor (antagonist) or it may have a mixed agonist/antagonist character.

The term “substantially corresponds” as used herein in relation to nucleotide sequences is intended to encompass minor variations in the nucleotide sequence which due to degeneracy in the DNA code do not result in a change in the encodied invertebrate 5-HT₃ receptor. Further, this term is intended to encompass other minor variations in the sequence which may be required to enhance expression in a particular system but in which the variations do not result in a decrease in biological activity of the encoded protein.

The term “substantially corresponding” as used herein in relation to amino acid sequences is intended to encompass minor variations in the amino acid sequences which do not result in a decrease in biological activity of the invertebrate 5-HT₃ receptor. These variations may include conservative amino acid substitutions. The substitutions envisaged are:

G, A, V, I, L, M; D, E; N, Q; S, T; K, R, H; F, Y, W, H; and P, Nα-alkylamino acids.

The terms “comprise”, “comprises” and “comprising” as used throughout the specification are intended to refer to the inclusion of a stated step, component or feature or group of steps, components or features with or without the inclusion of a further step, component or feature or group of steps, components or features.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

The invention will hereinafter be described with reference to the following non-limiting examples and accompanying figures.

### BRIEF DESCRIPTION OF THE ACCOMPANYING FIGURES

FIG. 1 provides dose-response curves for serotonin action on pharyngeal pumping. FIG. 1a is a dose response curve that was determined for serotonin by videotaping C. elegans under Nomarski optics and counting the rate of pharyngeal pumping over the range 325 μM-6.3 mM serotonin. FIG. 1b shows a dose response curve for the effect of serotonin on pharyngeal pumping rate obtained using a plate-based assay recently described in International Patent Application No. PCT/AU00/01476. Any minor differences between the curves of FIGS. 1a and 1b are accounted for by the differences in experimental detail and the fact that in the microscopical assay, the rate of pumping is counted over a period of 1-3 minutes, whilst in the plate-based assays the fluorescence intensity represents the accumulation of ingested material over 60 minutes.

FIG. 2 a typical dose-response curve for the inhibitory effect of MIDL72222 on the serotonin-stimulated pharyngeal pumping of C. elegans in the presence 0.325mM serotonin. The IC₅₀ of MDL 72222 in this experiment is approximately 25 μM (i.e. an order of magnitude) lower than the concentration of serotonin.

FIG. 3 provides a map of the molecular phylogeny of representative known mammalian 5-HT₃ sequences, representative known vertebrate and invertebrate nAChR sequences, the three putative receptor genes from ACeDB (F18G5.4.pep, F25G6.4/CE09640.pep and C31H5.3.pep) and the three related but distinct experimentally determined C. elegans 5-ET, sequences (F18, DIY and DIAY). This molecular phylogeny positions the three C. elegans 5-HT₃ receptor subunits in a framework created by a representative selection of mammalian 5-HT₃ receptor sequences (including the newly discovered 5HT_{3B}) and known nAChR sequences from an insect, nematode and human. The C. elegans genome project has assigned F18G5.4 as a hypothetical acetylcholine receptor like protein and also as a “ligand gated ion-channel”. The molecular phylogenetic analysis shows that, based on overall level of homology, F18G5.4 is the most similar of the available C. elegans sequences to previously identified mammalian 5-HT₃ receptors. The C. elegans genome project has assigned F25G6.4/CE09460 as a “ligand gated ion channel” and C31H5.3 as a putative 5HT₃ receptor. But the phylogeny of the present inventors shows that they are both as distantly related to currently known 5-HT, and nAChR as these two classes are to each other. On the other hand, the bold amino-acid triplets indicate the sequence found at the DIY position in each branch of the tree. It can be seen that both CE09460 and C31H5.3 are more similar to 5-HT_{3A} in respect of this character than are known nAChRs.

FIG. 4 shows the effects on serotonin-stimulated pharyngeal pumping of double-stranded RNA knockdown of the F18 messenger RNA in three separate and independent experiments. In each case, nematodes were fed according to Timmons. L. and Fire, A. (1998. Specific interference by ingested dsRNA. Nature. 395: 854) on HT115 E. coli bacteria expressing either a portion of the F18 gene “F18” or the empty double-stranded ds RNA expression plasmid “pL44440” as a control. In the third experiment (FIG. 4c), a second control comprising an irrelevant dsRNA being expressed from the same plasmid was also included “pCB6”. For each experimental treatment, individual nematodes are ordered along the X-axis according to their rank by pumping tate. Note that the concentration of serotonin used to challenge the nematodes is 3.25mM in FIGS. 4a and 4b and 1 mM in FIG. 4c. Other effects of RNAi using F18, in addition to the depressed pumping rate, are noted in Example 5 below.

For the experiments described below C. elegans of the Bristol N2 strain (Brenner, S. 1974. The genetics of Caenorhabditis elegans, Genetics, 77: 71-94) were cultured at room temperature on HMS174 E. coli bacteria (Campbell J. L. et al. 1978. Genetic recombination and complementation between bacteriophage T7 and cloned fragments of T77 DNA. Proc. Natl. Acad. Sci. USA. 75: 2276-2280) on NGM agar (Sulston, J. and Hodgkin, J. 1988, “Methods” in The nematode Caenorhabditis elegans. W. B. Wood pp 587-606. Cold Spring Harbor Laboratory Press, New York) unless otherwise described.

### EXAMPLE 1

### Pharmacological Identification of a Previously Unknown 5-HT₃ Receptor in C. elegans, Which is Responsible for Controlling the Rate and Strength of Pharyngeal Pumping

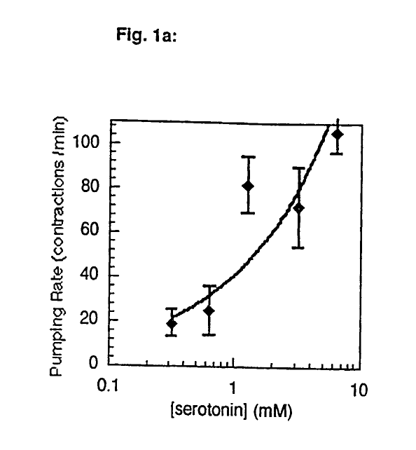

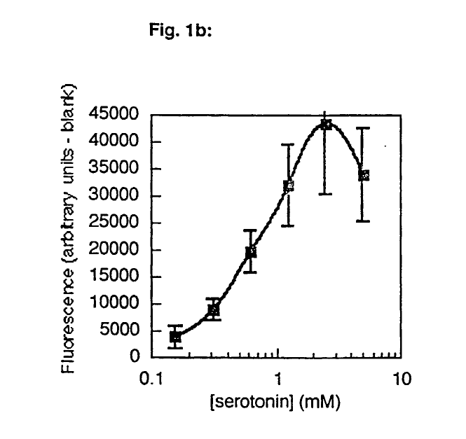

It is well-known that, among other effects, serotonin at micromolar to millimolar concentrations increases the rate and strength of nematode pharyngeal pumping (see FIG. 1. which shows dose response curves for serotonin acting on pharyngeal pumping).

5-HT₃-Receptor Class-Selective Azonists

Much is known about the pharmacology of vertebrate 5-HT receptors and there are a number of compounds in clinical use that act on the serotonergic system. Although relatively little is known about the pharmacology of invertebrate 5-HT receptors and there are some important differences from vertebrates, it seems that there is broad correspondence between the compounds that are active on vertebrate receptors of a given sub-class and invertebrate homologues.

Studies were therefore conducted wherein selective 5-HT₁, 5-HT₂ and 5-HT₃ agonists were applied at known concentrations to agar on which C. elegans was held in the absence of food and the effects on movement and pharyngeal pumping monitored over a period of 2-3 hours. The results for 5-HT₁, 5-HT₂ and 5-HT₃-selective agonists are shown in Table 1. A combination of two 5-HT₁ agonists each at 6 mM had little or no effect on pharyngeal pumping. Movement was inhibited but most nematodes recovered the ability to move after transfer to a fresh agar plate. A 5-HT₂ specific agonist also failed to stimulate pumping. A 5-HT₃-specific agonist (3 mM) caused a dramatic stimulation of pharyngeal pumping similar to a maximal concentration of 5-HT. There was no mortality of nematodes in any of the treatments during the time-course of the experiment.

Selective 5-HT₃ Antagonists.

Studies were also conducted to investigate the ability of selective vertebrate 5-HT₃ antagonists to inhibit the 5-HT-induced increase in pharyngeal pumping. The two inhibitors chosen were ondansetron (a Glaxo-Wellcome drug used to prevent chemotherapy-induced vomiting), which had been reported to be a much less potent inhibitor of the snail 5-HT₃-like ion channel than of the vertebrate 5-HT₃-receptor (IC₅₀ of 10 μM and 100 pM respectively) [Green, K. A. et al. 1996, supra], and MDL 72222 (tropanyl dichlorobenzoate) which had an IC₅₀ of 1 μM on the snail's 5-HT gated conductance [Green, K. A. et al. 1996, supra].

When the inhibitors were applied individually at 100 μM in the presence of 6.5 mM 5-HT they did not reduce the rate of pumping (not shown). However, 6.5 ml 5-HT is approximately 20× greater than the EC₅₀ (see FIG. 1) and so may out-compete inhibitors, particularly if they do not have very high affinity for the invertebrate receptor subtype. In a second experiment, the two inhibitors were applied together, each at a concentration of 325 μM and in the presence of 325 μM-5-HT (i.e. approximately the EC₅₀). There was total reversal of the 5-HT effect (see Table 2). In the absence of 5-HT, the effect of the two antagonists in combination was to stimulate pumping, although not to the same extent as 5-HT. In a third experiment, when the inhibitors were applied individually at an equimolar concentration with 5-HT (i.e. both 5-HT and the inhibitor at 0.325 mM) they each significantly inhibited the rate of pharyngeal pumping (Table 3). MDL72222, which on its own completely reversed the effect of the 5-HT, appeared to be significantly more effective than ondansetron under these conditions. Neither of the inhibitors on their own stimulated pharyngeal pumping.

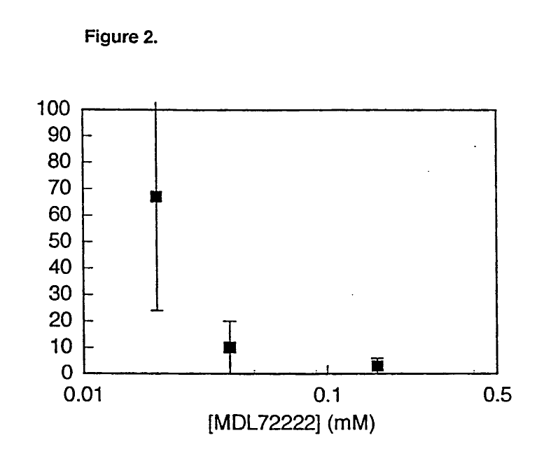

In another experiment, a dose response study was conducted to determine the effects of a range of doses of MDL72222 on the rate and strength of pharyngeal pumping in the presence of 0.325 mM serotonin (FIG. 2). At this concentration of 5-HT, the IC₅₀ for MDL72222 is approximately 30 μM, i.e. 10×lower than the EC₅₀ for 5-HT.

### EXAMPLE 2

### 5-HT₃-Selective Antagonist is Nematicidal for C. elegans and Insecticidal for the Sucking Insect Pest Myzus persicae (Hemiptera: Aphididae) and Antifeedant for the Chewing Insect Pest Helicoverpa armiera (Lepidoptera: Noctuidae).

The above data indicates that a 5-HT₃ receptor present in C. elegans and also likely to be present in other nematodes which respond to serotonin in a similar fashion to C. elegans and is responsible for mediating increased rate (and strength) of pharngeal pumping in response to exogenous or endogenous serotonin. Because the pharyngeal pump is essential for nematode feeding and the maintenance of the nematode “hydrostatic skeleton” [Brownlee, D. J. A. et al. 1997, supra] and a previously established target organ for nematicides, an investigation was conducted to assess whether the 5-HT₃-selective antagonists would function as nematicidal agents in a chronic exposure protocol.

C. elegans nematodes were cultured on the HMS/174 strain of E. coli [Campbell, J. L. et al. 1978. Genetic recombination and complementation between bacteriophage T7 and cloned fragments of T7 DNA. Proc. Natl. Acad. Sci. USA. 75: 2276-2280] on NGM agar [Sulston, J. and Hodgkin, J. 1988, supra] in the wells of six well tissue culture cluster, 2 ml agar per well. MDL72222 was incorporated into the agar at the specified concentrations using ≦65 μl of acetone as a carrier. A control well contained 65 μl of acetone only. Between five and 11 L1 stage C. elegans were transferred to each well and the wells were incubated at 22° C. for up to four weeks. Results are summarised in Table 4. All concentrations >10 μM MDL 72222 caused developmental delays. Concentrations of MDL 72222 down to 80 μM caused 40 % mortality within three days. At these concentrations, the nematodes died within a week and almost all their progeny were unable to hatch from the eggs. The bacterial food therefore remained uneaten for prolonged periods.

Concentrations of MDL72222 down to 20 μM caused 90-100% mortality within 21 days and concentrations as low as 10 μM and 5 μM caused 50-100% mortality at 21 days. At the lowest concentrations of MDL72222, nematode death was delayed until after the bacterial food had been consumed. At these concentrations, there were still clear differences from the nematodes in the control wells, which formed dauer larvae on exhaustion of the food supply, whereas the nematodes exposed to even the lowest concentrations of MDL72222 failed to respond properly to exhaustion of the food supply and died. The experiment shown in Table 3 was repeated with essentially identical results.

Because of the nematicidal efficacy of the 5-HT₃ selective antagonist against C. elegans, 5-HT₃-selective antagonists were tested to determine whether they would have a similar effect on two species of insect pest, namely Helicoverpa armigera and Myzus persicae. When incorporated into artificial diet (Teakle, R. E. and Jensen, J. M. 1985. Heliothis punctiger, in Handbook of insect rearing. P. Singh and R. F. Moore, Vol. 2. pp 312-322, Elsevier. Amsterdam), by surface contamination, MDL 72222 had a mild antifeedant effect on the growth and development of neonate H. armigera, which have biting and chewing mouthparts (Table 5). Ondansetron had no discernible effect of H. armigera (not shown).

The effects of MDL 72222 on Green Peach Aphid (Myzus persicae) was also tested. The bioassay involved testing compounds at 3 different concentrations (1 mM, 100 μM, 10 μM) in an artificial diet. Compounds that were not water-soluble were forced into solution using a non-toxic surfactant. For each compound, each concentration was tested in 10 replicate assays. In each replicate, 5 aphids were allowed to feed on the test solution in a Parafilm® sachet for a period of 5 days. Survivorship and aphid growth rate were compared among the test concentrations and a control containing only the artificial diet with surfactant. MDL 72222 caused dose-dependent mortality and significant reduction in weight gain in M. persicae (Table 6). Ondansetron had no discernible effect on M. persicae (not shown).

It is clear that MDL 72222 is a more potent inhibitor of the invertebrate 5-HT₃ receptor than is ondansetron.

It may, therefore, be concluded that agents that inhibit 5-HT₃ receptors of nematodes and those insects that have a feeding mechanism that involves a muscular pump (and therefore other invertebrates with similar mechanisms of feeding, maintaining their turgor pressure, or indeed attaching to their host by oral suction), will be universally pesticidal in those species. It can also be concluded that the 5-HT₃ receptors and subunits thereof from nematodes and other invertebrates represent ideal molecular targets for the screening, identification and optimisation of new, highly potent and highly invertebrate-selective pesticides.

### EXAMPLE 3

### Sequence Analysis

1. It is well known that vertebrate aminergic receptors of the 7-transmembrane G-protein-linked superfamily have a DRY triplet of amino acids located close to one end of the 3rd transmembrane segment. Analyses conducted by the present inventors have shown that 5-HT_{3A} receptors from four vertebrate species have a DIY conserved triplet.

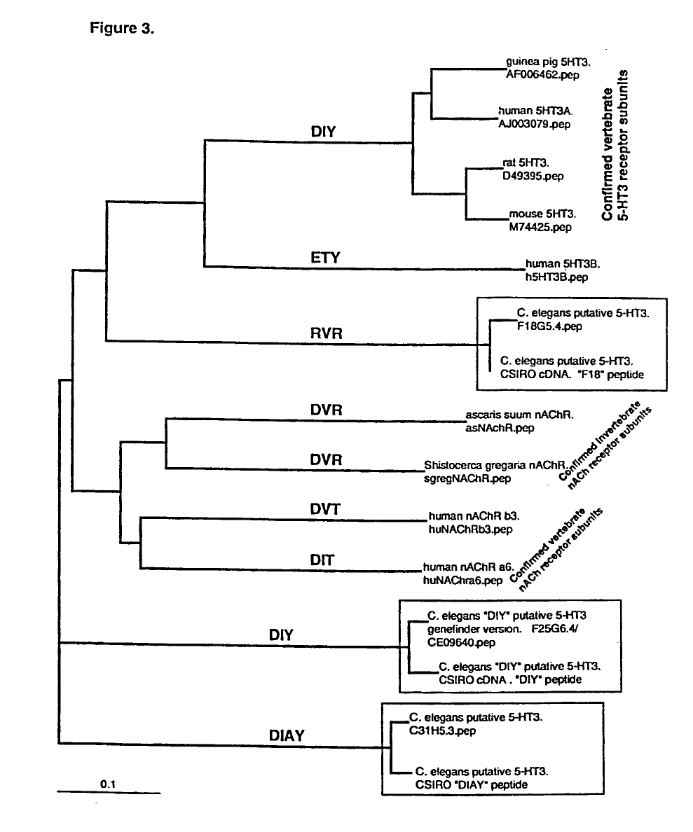

2. In an eclustalw (FIG. 3) alignment of representative known mammalian 5-HT₃ receptors, representative known mammalian, insect and nematode nAChRs and the three nematode 5-HT₃ receptor sequences of the present invention (previously assigned as hypothetical acetylcholine-like receptor F18G5.4, putative 5-HT₃ receptor C31H5.3 and ligand gated ion channel F25G6.4/CE09640), the latter three fall outside either of the known sub-classes of mammalian 5-HT₃ receptor subunits. However, F18G5.4 is the closest sequence to the group of mammalian 5-HT₃ receptors, F25G6.4/CE09640 has a DIY sequence motif in the expected position, while C31H5.3 has a DIAY (SEQ ID NO: 13) modified motif in the same position.

### EXAMPLE 4

### Cloning and Construction of Expression Cassettes

The three sequences described in Example 3 above were identified in the C. elegans genome database by the automated algorithm “GeneFinder”. Since it is not therefore possible to conclude that these sequences are-full-length or authentic, nor that they are expressed in vivo in the form identifed by GeneFinder, the present inventors cloned and expressed the three cDNAs (DIY, F18 and DIAY) which are encoded in part by the sequences identified as F25G6.4/CE09640. F18G5.4 and C31H5.3 in the C. elegans genome database.

1. Oligo-dT-primed cDNA was prepared from mixed life-stages of C. elegans. One portion of this was cloned into lambda gt10 to generate a cDNA library. Another portion was used as template for amplification of sequence-specific probes for each of the target genes. In each case, this was achieved using a pair of unique sequence primers designed, on the basis of the cDNAs predicted by GeneFinder, to span a central region (0.5-1.0 kb) of three predicted cDNAs. Although it would have been theoretically possible for this process to have failed due to errors in the GeneFinder predictions, subsequent sequencing of the amplified products indicated that in all three cases, authentic fragments of the desired target sequences were obtained.

2. ³²P-labelled probes were prepared from the amplicons by random-priming and the resultant probes were used to screen and recover hybridising cDNA clones by standard plaque lift using high stringency hybridisation.

3. For DIY, a single large clone was recovered from the library. However, sequencing revealed that this contained a splice error which introduced a number of in-frame stop codons. Two independent PCR amplicons were recovered from independent cDNA library pools, and a cDNA encoding the predicted coding sequence was reconstituted by ligation of a PCR fragment to the middle of the cDNA clone, thereby eliminating the splice error.

4. For DIAY, two independent large clones were recovered from the library. Sequencing and comparison to the ACeDB genome sequence indicated that these were both authentic clones bearing the complete coding sequence.

5. For F18, a single large clone was obtained from the library and several shorter clones which confirmed the 3' sequence. However, the longer sequence did not encode a plausible cleavable signal peptide at the 5' end. A search for additional sequences covering this region was instituted using anchored PCR with two nested F18-specific primers at the 3' end and a single lambda-phage-specific primer at the 5' end. Using independent pools from the cDNA library as template, two independent but identical amplicons were recovered which. when sequenced, predicted a plausible cleavable N-terminal signal peptide. A sequence encoding the predicted coding sequence was reconstructed by ligation of a fragment of one of the PCR amplicons to the 5' end of the cDNA clone.

The authenticity of the experimentally-determined clones was confirmed by double-stranded sequencing and analysis of the sequence as follows:

Comparison of the cDNA sequence with the C. elegans genoric sequence.

Presence of an open reading frame 1.5-2 kb.

Predicted membrane topology for a subunit of a ligand-gated ion-channel.

Predicted N-terminal cleavable signal peptide.

Sequence confirmed by multiple independent clones, either recovered from the cDNA library by screening and/or by PCR from independent cDNA pools.

In each case, experimental cloning and sequencing of the cDNAs. revealed errors in the GeneFinder predictions of the three cDNAs, especially in the assignment of intron-exon boundaries. The authentic isolated cDNA corresponding in part to ACeDB predicted gene CE09640/F25G6.4 has been designated “DIY” (SEQ ID NO: 5). The authentic reconstructed cDNA corresponding in part to the GeneFinder predicted cDNA C31H5.3 has been designated “DIAY” (SEQ ID NO: 6). Also, the authentic reconstructed cDNA corresponding in part to GeneFinder predicted cDNA F18G5.4 has been designated “F18” (SEQ ID NO: 4).

Each of the three cDNAs (DIY, F18 and DIAY) was inserted into the mammalian cell expression vector pcDNA3.1 (In-Vitrogen, Groningen, The Netherlands) to allow transfection of COS-7L cells and expression of each receptor subunit onto the cell surface. Each of the three cDNAs was also inserted into the related vector pcDNA3.1/myc-His (In-Vitrogen, Groningen, The Netherlands) to enable their expression as fusion proteins upstream of the C-myc and 6-His tags. Fusion proteins of this type have been shown not to interfere with expression of the mammalian ⁵-HT_{3A} subunit in HEK cells (Lankiewicz, S. et al., 2000. Phosphorylation of the 5-hydroxytryptamine (5-HT₃) receptor expressed in HEK293 cells. Receptors and Channels. 7: 9-15) (also related LGIC receptor subunits in COS-7L cells: Johnson and Trowell, unpublished observations). Correct in-frame construction of these expression vectors was confirmed by sequencing.

### EXAMPLE 5

### Functional Knockdown of F-18 and DIAY Using the RNAi Technique

For each of the cDNAs F18, DIY and DIAY, the present inventors amplified a central portion of the cDNA of between 0.5-1.0 kb using unique sequence PCR primers. The amplicon was ligated into the multiple cloning site of the plasmidic dsRNA expression vector pL4440 (Fire, A. et al. 1998. Patent and specific genetic interference by double-stranded RNA in Caenorhabditis elegans. Nature, 391. 806-8111 and the plasmid was transfected into the HT115 host strain of E. coli. HT115, is genetically deleted for double-stranded ribonuclease and contains a T7 RNA polymerase under the control of an IPTG(isopropyl thiogalactoside)-inducible promoter so that induction of the transfected bacteria with IPTG results in high level expression of the amplicon in the form of ds RNA.

Axenic C. elegans L1 stage were prepared by alkaline hypochlorite treatment of adult worms [Sulston J. and Hodgkin. J. 1988, supra] and were introduced onto an NGM agar petri dish with IPTGinduced bacteria expressing dsRNA from the gene of interest The nematodes were allowed to develop to the adult stage and were challenged with serotonin and the effects on the rate and strength of pharyngeal pumping were recorded by videomicroscopy.

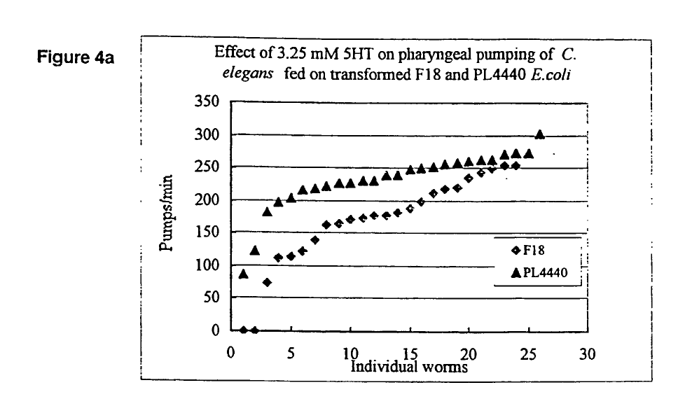

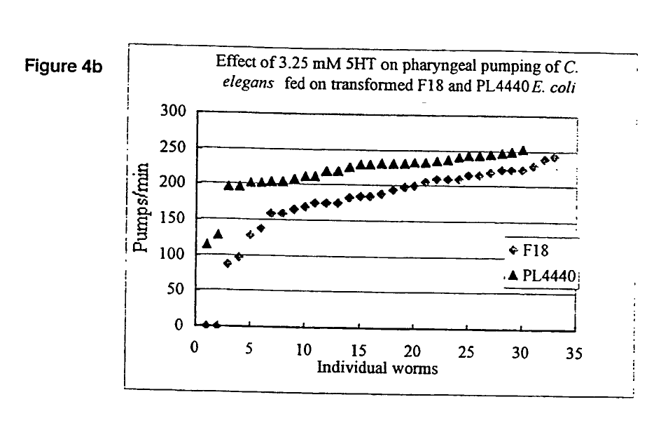

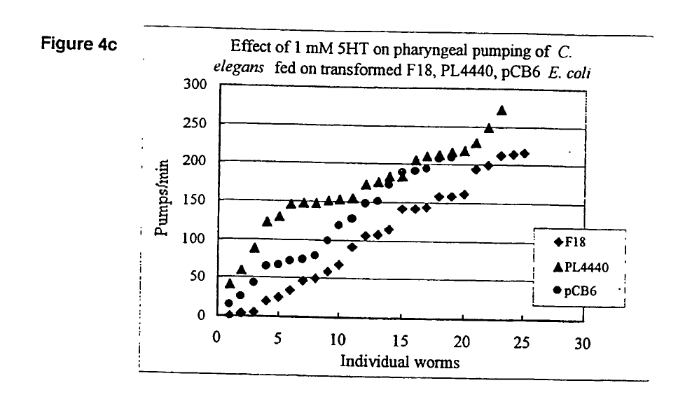

In three independent experiments, it was observed that F18 dsRNA caused a depression in both the rate of serotonin-stimulated pharyngeal pumping (FIG. 4 and Table 7) and the degree of opening/strength of contraction of the terminal bulb (Table 7). In the third experiment, the effectiveness of feeding of the nematodes was also assessed by challenging individual nematodes with serotonin in the presence of fluorescent beads and then observing the extent of bead ingestion. There was a visibly lower number of beads in the guts of nematodes exposed to the F18 dsRNA than in controls and the beads did not penetrate as far into the guts of the former.

Taken together, these results indicate that functional knockdown of F18 gene had a specific effect on pharyngeal pumping very similar to the effect of the 5-HT₃ specific antagonist MDL72222 and strongly indicates that F18 encodes a subunit of the nematode 5-HT₃ receptor.

The major difference in the effect was that, whereas MDL72222 completely suppresses pumping in all nematodes tested, in the case of the functional knockdown only a small number of worms were completely prevented from pumping, although the average pumping rate of the whole population was below that of the control (FIG. 4). The technique of RNAi knockdown by feeding is known to lack complete penetrance especially in neurones [Schuske, K. et al. 2000. Snap-back RNA: in neurons. The Worm Breeders' Gazette. 16: 18], and to be critically dependent on the induction protocol [Kamath, R. S. et al. 2000. Effectiveness of specific RNA-mediated interference through ingested double-stranded RNA in Caenorhabditis elegans. Genome Biology. 2: 1-10]. Therefore, to establish the degree of suppression of the F18 that had been achieved, individually identified worms from the F18 RNAi population and the control pL4440 plasmid population, whose rate of pumping had previously been established, were pooled in groups of three and subjected to quantitative reverse transcriptase PCR in a Corbett Research Rotorgene thermal cycler so as to measure the levels of F18 RNA. Comparison with standards indicated that even in control worms, the abundance of F18 mRNA is below that which can be quantified in three worms or fewer. However, it was possible to detect F18 message in all samples, indicating that suppression of F18 mRNA was not absolute.

One other difference that was noted between the F18 knockdown and normal worms was that there was a high incidence of visible anatomical defects in the pharynxes of the former. Preliminary high resolution microscopy indicates that this may be due to hypertrophy and degeneration of fine processes of the NSM motoneurones.

TABLE 1
Effects of 5-HT₃-receptor class-selective
agonists on C. elegans pharyngeal pumping and locomotion.

| | | | Pumping rate | |
|---|---|---|---|---|
| | Subclass- | | (min-1) | |
| Agonist | selectivity | Conc (mM) | mean ± s.d. | Effect on locomotion |
| No agonist | — | — | 5 ± 7 | |
| 5-carboxamido-tryptamine | 5HT_{1A · 1B · 1D} | 6 | 0 | Locomotion inhibited |
| maleate (Tocris TC0458) + | | | | |
| BRL-54443 (Tocris TC1129) | 5-HT_{1E · 1F} | 6 | | |
| 5-carboxamido-tryptamine | 5HT_{1A · 1B · 1D} | 1 | 0 | Locomotion normal |
| maleate (Tocris TC0458) + | | | | |
| BRL-54443 (Tocris TC1129) | 5-HT_{1E · 1F} | 1 | | |
| m-CPP hydrochloride | 5-HT_{2B · 2C} | 6 | 0 | Locomotion inhibited |
| (TocrisTC0875) | | 1 | 0 | Locomotion inhibited |
| 2-methyl-5- | 5-HT₃ | 3 | 130 ± 27 | Locomotion normal, |
| hydroxytryptamine | | | | or faster than normal |
| hydrochloride (Tocris | | | | |
| TC0558) | | | | |

TABLE 2
Effects of two 5-HT₃-receptor class-selective
antagonists in combination on
C. elegans pharyngeal pumping
and locomotion.

| | Pumping rate | |
|---|---|---|
| | (min-1) | |
| Treatment | mean ± s.d. | Effect on locomotion |
| Control, no addition | 10 ± 10 | Nematodes moved normally |
| 0.325 mM serotonin | 129 ± 6 | Nematodes moved normally |
| 0.325 mM serotonin plus | 5 ± 5 | Nematodes did not move |
| 0.325 mM ondansetron and | | |
| 0.325 mM MDL 72222 | | |
| 0.325 mM ondansetron and | 46 ± 73 | Nematodes moved normally |
| 0.325 mM MDL 72222 | | |

TABLE 3
Effects of individual 5-HT₃-receptor
class-selective antagonists on
C. elegans pharyngeal pumping and
locomotion.

| | Pumping rate | |
|---|---|---|
| | (min-1) | |
| Treatment | mean ± s.d. | Effect on locomotion |
| Control, no addition | 8 ± 7 | Nematodes moved normally |
| 0.325 mM serotonin | 54 ± 45 | Nematodes moved normally |
| 0.325 mM serotonin plus | 32 ± 29 | Nematodes moved normally |
| 0.325 mM ondansetron | | |
| 0.325 mM serotonin plus | 2 ± 2 | >50% of nematodes did not |
| 0.325 mM MDL 72222 | | move unless stimulated |
| 0.325 mM ondansetron | 0 ± 0 | Nematodes moved normally |
| 0.325 mM MDL 72222 | 1 ± 1 | >50% of nematodes did not |
| | | move unless stimulated |

TABLE 4
Effects of MDL-72222, a 5-HT₃-receptor
class-selective antagonist,
on nematode growth and survival.

| | Number of | % | % | Time to |
|---|---|---|---|---|
| [MDL72222] | worms | Mortality - | Mortality - | consumption of |
| (μM) | seeded | 3 days | 21 days | all bacteria (days) |
| 325 | 9 | 55 | 100 | >28 |
| 162 | 11 | 64 | >90 | >28 |
| 81 | 7 | 42 | >90 | 16 |
| 40 | 9 | 0 | >90 | 10 |
| 20 | 8 | 0 | 100 | 9 |
| 10 | 8 | 0 | >50 | 8 |
| 5 (carrier) | 7 | 0 | 100 | 7 |
| 0 (acetone carrier) | 7 | 0 | <10 | 7 |
| 0 (water) | 5 | 0 | <10 | 8 |

TABLE 5
Effects of MDL-72222, a 5-HT₃-receptor
class-selective antagonist, on growth
of H. armigera over 7 days. 24
larvae in each treatment group.
Because the larvae were weighed en masse
to derive the mean weight an estimate of
variance is unavailable.

| | Mean weight | |
|---|---|---|
| Treatment (by surface | of larva | |
| contamination of diet) | (mg) at 7 days | % of control weight |
| Control, water | 73 | 100 |
| Control, acetone | 71 | 99 |
| 0.1 mM serotonin in water | 85 | 116 |
| 1.0 mM serotonin in water | 85 | 116 |
| 0.1 mM MDL 72222 in acetone | 51 | 70 |
| 1.0 mM MDL 72222 in acetone | 54 | 75 |

TABLE 6
Effects of MDL-72222, a 5-HT₃-receptor
class-selective antagonist, on
growth and survival of Myzus persicae
after 5 days.

| | Mean number of | |
|---|---|---|
| | surviving aphids | Mean relative growth |
| Treatment | (out of 5) | rate of survivors |
| Control, diet and carrier only | 5.0 | 0.0549 |
| 1 mM MDL 72222 | 0.9 | 0.0383 |
| 0.1 mM MDL 72222 | 3.4 | 0.0200 |
| 0.01 mM MDL 72222 | 4.6 | 0.0339 |

TABLE 7
Summary of effects of RNAi-mediated gene
knockdown with a portion of the F18 cDNA.
Effects on rate of pumping and
the degree of grinder opening are shown.

| | | | | % grinder |
|---|---|---|---|---|
| | No. of worms | Mean. Pumps | | opening |
| RNAi gene | tested | (min-1) | StD | fully |
| Expt 1: | | | | |
| F18 | 24 | 169 | 15 | 46 |
| No insert control | 26 | 232 | 9 | 100 |
| Expt 2: | | | | |
| F18 | 33 | 177 | 10 | 55 |
| No insert control | 30 | 219 | 6 | 97 |
| Expt 3: | | | | |
| F18 | 25 | 107 | 15 | 42 |
| No insert control | 23 | 169 | 12 | 100 |
| Irrelevant insert | 19 | 119 | 16 | 100 |
| control | | | | |

1. An isolated polynucleotide molecule encoding an invertebrate 5-HT₃ receptor subunit consisting of a nucleotide sequence which shows greater than 75% homology to any or all of the nucleotide sequences shown as SEQ ID NO: 1-6.

2. A polynucleotide molecule according to claim 1, wherein said polynucleotide molecule consists of a nucleotide sequence which shows greater than 85% homology to any or all of the nucleotide sequences shown as SEQ ID NO: 1-6.

3. A polynucleotide molecule according to claim 1, wherein said polynucleotide molecule consists of a nucleotide sequence which shows greater than 95% homology to any or all of the nucleotide sequences shown as SEQ ID NO: 1-6.

4. A polynucleotide molecule according to claim 1, wherein said polynucleotide molecule consists of a nucleotide sequence which substantially corresponds to any one of the nucleotide sequences shown as SEQ ID NO: 1-6.

5. A polynucleotide molecule according to claim 1, wherein said polynucleotide molecule consists of a nucleotide sequence which substantially corresponds to the nucleotide sequence shown as SEQ ID NO: 1.

6. A polynucleotide molecule according to claim 1, wherein said polynucleotide molecule consists of a nucleotide sequence which substantially corresponds to the nucleotide sequence shown as SEQ ID NO: 2.

7. A polynucleotide molecule according to claim 1, wherein said polynucleotide molecule consists of a nucleotide sequence which substantially corresponds to the nucleotide sequence shown as SEQ ID NO: 3.

8. A polynucleotide molecule according to claim 1, wherein said polynucleotide molecule, consists of a nucleotide sequence which substantially corresponds to the nucleotide sequence shown as SEQ ID NO: 4.

9. A polynucleotide molecule according to claim 1, wherein said polynucleotide molecule consists of a nucleotide sequence which substantially corresponds to the nucleotide sequence shown as SEQ ID NO: 5.

10. A polynucleotide molecule according to claim 1, wherein said polynucleotide molecule consists of a nucleotide sequence which substantially corresponds to the nucleotide sequence shown as SEQ ID NO: 6.

11. An expression cassette or vector comprising the polynucleotide molecule according to any one of the preceding claims.

12. A mammalian, insect, plant, yeast or bacterial host cell transformed with at least one expression cassette or vector according to claim 11.

13. A host cell according to claim 12, wherein said host cell expresses homomeric invertebrate 5-HT₃ receptors.

14. A host cell according to claim 12, wherein said host cell expresses heteromeric invertebrate 5-HT₃ receptors.

15. A host cell according to claim 13 or 14. wherein said 5-HT₃ receptors are expressed onto the surface of the host cell.

16. A method of producing 5-HT₃ receptors, comprising culturing a host cell according to any one of claims 11-15 under conditions enabling the expression of said 5-HT₃ receptors and, optionally, recovering the expressed 5-HT₃ receptors.

17. An invertebrate 5-HT₃ receptor comprising at least one subunit which is characterised by an N-terminal amnino acid sequence selected from the following:
MIICYSCLTV (SEQ ID NO: 7), MLLPILLHFL (SEQ ID NO: 8) or MRRRFEIGIA (SEQ ID NO: 9),
or a functionally equivalent fragment of said receptor, in a substantially pure form.

18. A receptor according to claim 17, wherein the at least one subunit has an amino acid sequence substantially corresponding to that shown as SEQ ID NO: 10, 11 or 12.

19. An assay for identifying and/or assessing nematicidal compounds, said assay comprising contacting a 5-HT₃ receptor or a functionally equivalent fragment thereof according to claim 17 or 18, or a host cell according to claim 15 having 5-HT₁₃ receptors on its surface. with a candidate nematicidal compound under conditions enabling the activation of 5-HT receptors, and detecting an increase or decrease in activity of said 5-HT₃ receptor(s) or functionally equivalent fragment thereof.

20. An assay according to claim 19, wherein the increase or decrease in activity of the 5-HT₃ receptor(s) or functionally equivalent fragment thereof is detected by measuring changes in cell membrane potential or Ca²⁺ levels.

21. An assay according to claim 19 or 20, wherein the the 5-HT₃ receptor(s) or functionally equivalent fragment thereof is contacted simultaneously with the candidate nematicidal compound and a serotonergic ligand.

22. An assay for identifying and/or assessing nematicidal compounds, said assay comprising contacting a 5-HT₃ receptor or a functionally equivalent fragment thereof according to claims 17 or 18, or a cell according to claim 15 having 5-HT₃ receptors on its surface, with a predetermined amount of a suitably labelled serotonergic ligand together with a predetermined amount of a candidate nematicidal compound under conditions wherein said serotonergic ligand and said candidate nematicidal compound competitively bind to the 5-HT₃ receptor(s) or functionally equivalent fragment thereof, and determining the amount of bound and/or unbound labelled serotonergic ligand.

23. An assay according to any one of claims 19 to 22, wherein said serotonergic ligand is 5-hydroxytryptamine.

24. A method for identifying a polynucleotide sequence encoding a 5-HT₃ receptor subunit, the method comprising exposing a candidate polynucleotide to a sequence which comprises at least 10 nucleotides of a nucleotide sequence as shown in SEQ ID NO: 1-6.

25. The method of claim 24, wherein the 5-HT₃ receptor subunit is isolated from an invertebrate.

26. An assay for identifying and/or assessing nematicidal, insecticidal and/or other pesticidal compounds, said assay comprising;
(i) isolating a polynucleotide molecule from an invertebrate other than Caenorhabditis elegans wherein said polynucleotide molecule comprises a nucleotide sequence which encodes a 5-HT₃ receptor subunit and represents a homologue of any or all of the nucleotide sequences shown as SEQ ID NO: 1-6,
(ii) expressing said polynucleotide molecule to produce 5-HT₃ receptors or functionally equivalent fragments thereof,
(iii) contacting at least one of said produced 5-HT₃ receptors or functionally equivalent fragments thereof to a candidate nematicidal, insecticidal and/or other pesticidal compound under conditions enabling the activation of 5-HT₃ receptors, and
(iv) detecting an increase or decrease in activity of said produced 5-HT₃ receptor(s) or functionally equivalent fragment(s) thereof.

27. An assay according to claim 26, wherein the increase or decrease in activity of the 5-HT₃ receptor(s) or functionally equivalent fragment thereof is detected by measuring changes in cell membrane potential or Ca²⁺ levels.

28. An assay according to claim 26 or 27, wherein the 5-HT₃ receptor(s) or functionally equivalent fragment thereof is contacted simultaneously with the candidate nematicidal compound and a serotonergic ligand.

29. An assay for identifying and/or assessing nematicidal, insecticidal and/or other pesticidal compounds, said assay comprising;
(i) isolating a polynucleotide molecule from an invertebrate other than Caenorhabditis elegans wherein said polynucleotide molecule comprises a nucleotide sequence which encodes a 5-HT₃ receptor subunit and represents a homologue of any or all of the nucleotide sequences shown as SEQ ID NO: 1-6,
(ii) expressing said polynucleotide molecule to produce 5-HT₃ receptors or functionally equivalent fragments thereof,
(iii) contacting at least one of said produced 5-HT₃ receptors or functionally equivalent fragments thereof with a predetermined amount of a suitably labelled serotonergic ligand together with a predetermined amount of a candidate nematicidal, insecticidal and/or other pesticidal compound under conditions wherein said serotonergic ligand and said candidate compound competitively bind to the 5-HT₃ receptor(s) or functionally equivalent fragment(s) thereof, and
(iv) determining the amount of bound and/or unbound labelled serotonergic ligand.

30. An assay according to any one of claims 26 to 29, wherein said serotonergic ligand is 5-hydroxytryptamine.

31. A nematicidal compound identified by an assay according to any one of claims 19 to 23.

32. A nematicidal, insecticidal and/or other pesticidal compound identified by an assay according to any one of claims 26 to 30.

33. A method of killing a helminth, said method comprising exposing said helminth to an effective amount of a compound which alters the activity of a 5-HT₃ receptor of said helminth.

34. A method according to claim 33, wherein said compound inhibits stimulation of the 5-HT₃ receptor by a serotonergic ligand.

35. A method according to claim 33 or 34, wherein said effective amount of said compound is in the range of 0.1 to 10 μM.

36. A method according to any one of claims 33 to 35, wherein the helminth is a nematode.

37. A method according to any one of claims 33 to 36, wherein said compound is selected from ondansetron and tropanyl dichlorobenzoate.

38. A composition for killing a helminth comprising an effective amount of a compound which alters the activity of a 5-HT₃ receptor of a helminth, wherein said compound is not ondansetron or tropanyl dichlorobenzoate.

39. A composition according to claim 38, wherein said compound inhibits stimulation of the 5-HT₃ receptor by a serotonergic ligand.

40. A composition according to claim 38 or 39, wherein said effective amount of said compound is in the range of 0.1 to 10 μM.

41. A composition according to any one of claims 38 to 40, wherein the helminth is a nematode.

42. A method of killing an insect, said method comprising exposing said insect to an effective amount of a compound which alters the activity of a 5-HT₃ receptor of said insect.

43. A method according to claim 42, wherein said compound inhibits stimulation of the 5-HT₃ receptor by a serotonergic ligand.

44. A method according to claim 42 or 43, wherein said effective amount of said compound is in the range of 0.1 to 10 μM.

45. A method according to any one of claims 42 to 44, wherein the insect is a sucking insect or other insect with a muscular pump-based feeding mechanism.

46. A method according to any one of claims 42 to 45, wherein said compound is selected from ondansetron and tropanyl dichlorobenzoate.

47. An insecticidal composition comprising an effective amount of a compound which alters the activity of a 5-HT₃ receptor of an insect, wherein said compound is not ondansetron or tropanyl dichlorobenzoate.

48. A composition according to claim 47, wherein said compound inhibits stimulation of the 5-HT₃ receptor by a serotonergic ligand.

49. A composition according to claim 47 or 48, wherein said effective amount of said compound is in the range of 0.1 to 10 μM.

50. A composition according to any one of claims 47 to 49, wherein the insect is a sucking insect or other insect with a muscular pump-based feeding mechanism.

51. An isolated polynucleotide molecule from an invertebrate other than Caenorhabditis elegans, wherein said polynucleotide molecule comprises a nucleotide sequence which encodes a 5-HT₃ receptor subunit and represents a homologue of any or all of the nucleotide sequences shown as SEQ ID NO: 1-6.
